(19) United States
(12) Patent Application Publication
(10) Pub. No.: US 2014/0308268 A1
(43) Pub. Date: Oct. 16, 2014

(54) DETERGENT COMPOSITION

(71) Applicant: Wen-Chen WEI, Taichung City (TW)

(72) Inventor: Wen-Chen WEI, Taichung City (TW)

(21) Appl. No.: 14/102,636

(22) Filed: Dec. 11, 2013

(30) Foreign Application Priority Data
Apr. 10, 2013 (TW) 102112644

(51) Int. Cl. A61K 36/18; A61K 36/889

(52) U.S. Cl. CPC A61K 36/18; A61K 36/889
USPC 424/115

(57) ABSTRACT

A detergent composition includes an alcoholic beverage, a moisturizer, a surfactant and water; and the surfactant is a natural coconut oil foaming agent. By including the alcoholic beverage, the detergent composition does not need any addition of a preserving agent and provides additional disinfecting effect as well as upgraded cleansing effect.

### FIELD OF THE INVENTION

The present invention relates to a detergent composition, and more particularly, to a detergent composition that does not need any addition of preserving agent and provides additional disinfecting effect as well as upgraded cleansing effect.

### BACKGROUND OF THE INVENTION

In people's daily life, various kinds of detergents and washing agents are almost requisite items for maintaining personal hygiene and environmental cleanness. Detergents for different purposes, such as clothes washing, bathroom and kitchen cleaning, dish washing, vegetable and fruit washing and the like, have their own special compositions to satisfy consumers' different requirements.

Taking dish washing as an example, most consumers often purchase detergents to wash their dishes without knowing the exact compositions of the detergents. There are times bitter and acerbic tasted chemical compounds of the purchased detergents remained on the cleaned dishes. The consumers are subject to dangers of health if they are in oral contact with such remained chemical compounds over a long period time.

In response to consumers' requirements for body health and environmental and ecological protection, detergent manufacturers, particularly dish washing agent manufacturers, have constantly researched to search for and develop detergent compositions that are environmentally friendly and have good cleansing ability with low chemical residue. To this purpose, natural substances that have low chemical residue and do not threaten consumers' health have been considered as additives to dish washing agents.

The main ingredients of commercially available detergents for dish washing include surfactant, sodium sulfate, phosphate, preserving agent, thickener, spice and so on. The purpose of adding the preserving agent to the detergents is to prevent the products from becoming denatured. However, the preserving agent tends to remain on the dishes if the latter are not thoroughly rinsed with clean water, and enters users' bodies next time along with the food held in the dishes. The preserving agent accumulated in bodies will endanger people's health and also has negative influence on people's living environment.

The detergent manufacturers have tried to add some natural cleansing and disinfecting ingredients, such as eucalyptus extract and cypress extract, to the dish washing agents. However, the anti-bacteria property of these natural compounds is not as satisfactory as expected. In view that the natural anti-bacteria compounds are not satisfactory in their effect and the chemical substances with enhanced disinfecting effect tend to have high residue to endanger users' health and living environment, it is very important to work out a detergent composition that includes natural ingredients having low residue and good disinfecting effect and is therefore particularly suitable for dish washing.

In conclusion, the prior art detergent compositions have the following disadvantages: (1) harmful to human body and not environmentally friendly; (2) low cleaning efficiency; and (3) lacking disinfecting effect.

It is therefore tried by the inventor to develop an improved detergent composition to overcome the above problems and disadvantages.

### SUMMARY OF THE INVENTION

A primary object of the present invention is to provide a detergent composition that provides upgraded cleansing effect.

Another object of the present invention is to provide a detergent composition that provides good disinfecting effect.

To achieve the above and other objects, the detergent composition provided by the present invention includes an alcoholic beverage, a moisturizer, a surfactant and water; and the surfactant is a natural coconut oil foaming agent.

Since the alcoholic beverage containing alcohol provides disinfecting effect, the use of the detergent composition of the present invention including the alcoholic beverage in washing dishes can kill microorganisms left on the dishes. That is, it is not necessary to add any preserving agent to the detergent composition while the dishes can be thoroughly cleaned and disinfected.

### DETAILED DESCRIPTION OF THE PREFERRED EMBODIMENTS

The present invention relates to a detergent composition including an alcoholic beverage, a moisturizer, a surfactant, and water.

The alcoholic beverage can be any one of beer, wine and spirits. While the embodiments of the present invention are described with the alcoholic beverage being a type of spirits, it is understood any other alcoholic beverages that provide a disinfecting effect can be adopted for achieving the objects of the present invention.

Surfactants can be generally classified into four types, namely, anionic surfactants, cationic surfactants, zwitterion surfactants and nonionic surfactants. Any one of these surfactants contains water-soluble parts, i.e. hydrophilic groups, and water-insoluble but oil-soluble parts, i.e. hydrophobic groups. The surfactant adopted in the embodiments of the present invention is a zwitterionic surfactant, such as a natural coconut oil foaming agent. Zwitterionic surfactants have the features of both anionic surfactants and cationic surfactants. Generally speaking, zwitterionic surfactants as cleansing agents have the advantages of producing finer bubbles, providing proper oil-removing ability, being less irritating, and having moisturizing and disinfecting functions, and are therefore frequently added to detergents along with anionic surfactants to reduce the irritating nature of the anionic surfactants. However, it is understood the surfactant that can be adopted by the present invention is not limited to any specific type. Any ingredients that are functionally equivalent to the coconut oil foaming agent can be used in replace of the latter.

The moisturizer can be selected from any one of polyols, natural moisturizing factors, amino acids and biochemical polymers. However, it is understood, in practical implementation of the present invention, any other functionally equivalent moisturizing agents can also be used with the present invention.

According to a preferred embodiment of the present invention, the detergent composition thereof can further include rice extract, wheat extract, rice and wheat extracted essence, or a combination thereof. Rice and wheat are natural products. Wheat extract is harmless to a user's hands and provides high cleaning effect. Rice and wheat extracted essence gives the detergent composition an increased viscidity and can be used in place of palm oil, which is often used as an additive to the currently commercially available cleansing products. The fully natural plant extracts adopted by the present invention are completely bio-degradable and are environmentally friendly. Further, it has been proven in research and development experiments as well as clinical trial uses that the natural coconut oil foaming agent with rice extract, wheat extract or rice and wheat extracted essence added thereto is soluble in a stable and balanced manner and has no unpleasant odor.

Alcoholic beverages contain alcohol, which absorbs water in bacterioprotein left on not fully cleaned dishes to cause dehydrated, denatured and coagulated protein and thereby kill bacteria to achieve the purpose of disinfection. Therefore, alcoholic beverages can be used in place of other industrial or cosmetic purpose preserving agents usually added to the currently commercially available detergent products. That is, by adding an alcoholic beverage to the detergent composition of the present invention, microorganisms left on dishes can be killed when the dishes are washed with the alcohol-containing detergent composition of the present invention, making the dishes particularly cleaned and disinfected.

Since the detergent composition of the present invention includes fully natural ingredients, it can also be used to wash vegetables, fruits and other foodstuffs without the risk of causing harms to human body.

In another preferred embodiment of the present invention, a fragrant substance can be further added to the detergent composition according to actual need to produce some desired fragrance for the detergent composition. The fragrant substance can be a botanical spice or a natural spice.

In brief, compared to conventional detergents, the detergent composition according to the present invention has the following advantages: (1) completely non-toxic and harmless to human body; (2) high cleaning efficiency; (3) containing disinfecting ingredient; (4) hand-protective; and (5) no need for preserving agent.

The present invention has been described with some preferred embodiments thereof and it is understood that many changes and modifications in the described embodiments can be carried out without departing from the scope and the spirit of the invention that is intended to be limited only by the appended claims.

What is claimed is:

1. A detergent composition, comprising an alcoholic beverage, a moisturizer, a surfactant, and water.

2. The detergent composition as claimed in claim 1, wherein the surfactant is a natural coconut oil foaming agent.

3. The detergent composition as claimed in claim 1, wherein the moisturizer is selected from the group consisting of polyols, natural moisturizing factors, amino acids and biochemical polymers.

4. The detergent composition as claimed in claim 1, further comprising a rice extract.

5. The detergent composition as claimed in claim 1, further comprising a wheat extract.

6. The detergent composition as claimed in claim 1, further comprising a combined rice and wheat extracted essence.

7. The detergent composition as claimed in claim 1, further comprising a fragrant substance.

8. The detergent composition as claimed in claim 7, wherein the fragrant substance is selected from the group consisting of botanical spices and natural spices.

9. The detergent composition as claimed in claim 1, wherein the alcoholic beverage is a type of spirits.
